(19) United States
(12) Patent Application Publication
(10) Pub. No.: US 2015/0173721 A1
(43) Pub. Date: Jun. 25, 2015

(54) ULTRASOUND DIAGNOSTIC APPARATUS, MEDICAL IMAGE PROCESSING APPARATUS AND IMAGE PROCESSING METHOD

(71) Applicants: Kabushiki Kaisha Toshiba, Minato-ku (JP); Toshiba Medical Systems Corporation, Otawara-shi (JP)

(72) Inventors: Shunsuke SATOH, Nasushiobara (JP); Kazutoshi SADAMITSU, Otawara (JP); Takayuki GUNJI, Otawara (JP); Koichiro KURITA, Nasushiobara (JP); Yu IGARASHI, Utsunomiya (JP)

(73) Assignees: Kabushiki Kaisha Toshiba, Minato-ku (JP); Toshiba Medical Systems Corporation, Otawara-shi (JP)

(21) Appl. No.: 14/643,220

(22) Filed: Mar. 10, 2015

Related U.S. Application Data
(63) Continuation of application No. PCT/JP2013/074291, filed on Sep. 9, 2013.

(30) Foreign Application Priority Data
Sep. 10, 2012 (JP) 2012-198937
Sep. 9, 2013 (JP) 2013-186717

(51) Int. Cl. A61B 8/08; A61B 8/00

(52) U.S. Cl. CPC A61B 8/5246; A61B 8/463; A61B 8/5207

(57) ABSTRACT

An ultrasound diagnostic apparatus includes an alignment unit, a detector and a generator. The alignment unit performs alignment between three-dimensional ultrasound volume data and three-dimensional different-type medical image volume data of a type other than the three-dimensional ultrasound volume data. The detector specifies the position of a luminal area on the different-type medical image volume data and detects the specified position of the luminal area on the ultrasound volume data. The generator generates, as display image data to be displayed on a given display unit, projection image data obtained by projecting the ultrasound volume data from a viewpoint that is set in the luminal area on the basis of the position of the luminal area that is detected by the detector.

### CROSS-REFERENCE TO RELATED APPLICATIONS

This application is a continuation of International Application No. PCT/JP2013/074291, filed on Sep. 9, 2013 which claims the benefit of priority of the prior Japanese Patent Application No. 2012-198937, filed on Sep. 10, 2012 and Japanese Patent Application No. 2013-186717, filed on Sep. 9, 2013, the entire contents of which are incorporated herein by reference.

### FIELD

Embodiments described herein relate generally to an ultrasound diagnostic apparatus, a medical image processing apparatus and an image processing method.

### BACKGROUND

An ultrasound diagnostic apparatuses has superior ability in depicting a fine structure compared to other medical image diagnostic apparatuses, such as X-ray CT (Computed Tomography) apparatuses and MRI (Magnetic Resonance Imaging) apparatuses, and is, for example, a medical image diagnostic apparatus beneficial in observing the blood-vessel-based circulatory system. In recent years, ultrasound diagnostic apparatuses are in practical use that generates volume data approximately in real time in a chronological order by using an ultrasound probe capable of ultrasound three-dimensional scanning.

For this reason, in the field of ultrasound examination as well, introduction of virtual endoscopic display that is performed for volume data acquired by an X-ray CT (Computed Tomography) apparatus, an MRI (Magnetic Resonance Imaging) apparatus etc. has been promoted. For example, virtual endoscopic display of blood vessels by using an ultrasound diagnostic apparatus is beneficial as a new method of observing circulatory diseases, particularly, angiostenosis and aneurism. In order to perform virtual endoscopic display, it is required to detect a luminal area of the lumen contained in an ultrasound volume data (e.g., B-mode volume data).

However, in an ultrasound image (B-mode image), compared to other medical images, such as X-ray CT images and MRI images, the outline of structures are more likely to be blurred. Thus, unless the lumen has a certain diameter or more, it is difficult to detect a luminal area of the lumen from the B-mode volume data by automatic processing using a program. For this reason, currently, virtual endoscopic display in an ultrasound diagnostic apparatus is limited to tubular tissues with a certain diameter and is difficult to be applied to narrow tubular tissues.

### BRIEF DESCRIPTION OF THE DRAWINGS

FIG. 1 is a block diagram depicting an exemplary configuration of an ultrasound diagnostic apparatus according to a first embodiment;

FIG. 2 is a diagram for describing an exemplary configuration of a controller 17 according to the first embodiment;

FIG. 3 is a diagram for describing an alignment unit according to the first embodiment;

FIG. 4 is a diagram for describing an acquisition unit according to the first embodiment;

FIG. 5 is a diagram for describing the acquisition unit according to the first embodiment;

FIG. 6 is a diagram for describing a generator according to the first embodiment;

FIG. 7 is a diagram for describing the generator according to the first embodiment;

FIG. 8 is a flowchart for describing exemplary processing performed by the ultrasound diagnostic apparatus according to the first embodiment;

FIG. 9 is a diagram depicting other exemplary display image data;

FIG. 10 is a diagram depicting other exemplary display image data; and

FIG. 11 is a block diagram depicting an exemplary configuration of a medical image processing apparatus according to a second embodiment.

### DETAILED DESCRIPTION

An ultrasound diagnostic apparatus includes an alignment unit, a detector and a generator. The alignment unit performs alignment between three-dimensional ultrasound volume data and three-dimensional different-type medical image volume data of a type other than the three-dimensional ultrasound volume data. The detector specifies the position of a luminal area on the different-type medical image volume data and detects the specified position of the luminal area on the ultrasound volume data. The generator generates, as display image data to be displayed on a given display unit, projection image data obtained by projecting the ultrasound volume data from a viewpoint that is set in the luminal area on the basis of the position of the luminal area that is detected by the detector.

An ultrasound diagnostic apparatus, a medical image processing apparatus and an image processing method according to embodiments are described below with reference to the drawings.

### First Embodiment

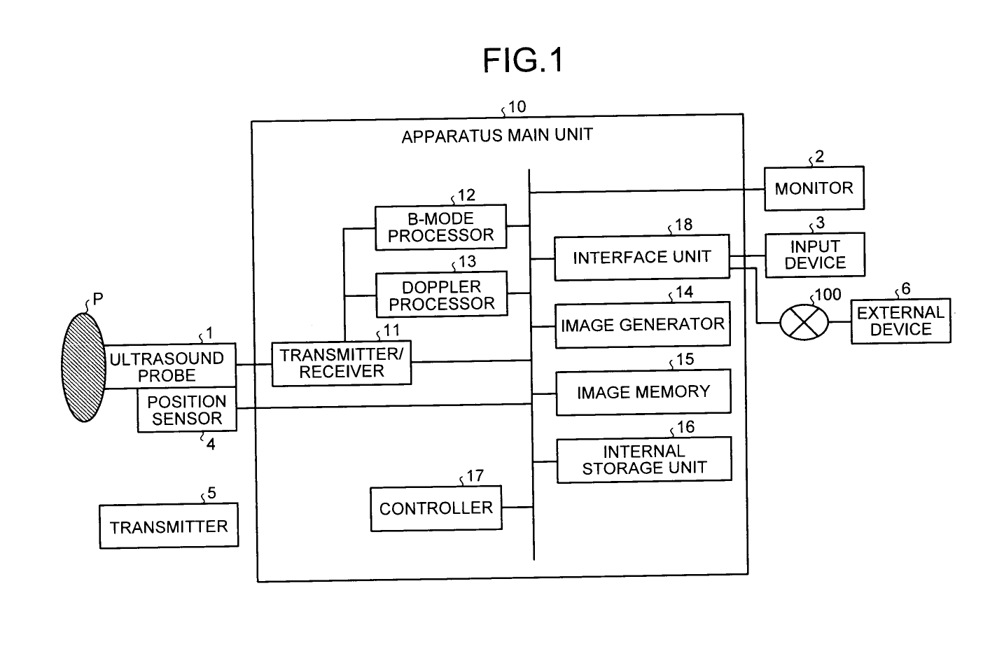

First, a configuration of an ultrasound diagnostic apparatus according to a first embodiment will be described. FIG. 1 is a block diagram depicting an exemplary configuration of the ultrasound diagnostic apparatus according to the first embodiment. As illustrated in FIG. 1, the ultrasound diagnostic apparatus according to the first embodiment includes an ultrasound probe 1, a monitor 2, an input device 3, a position sensor 4, a transmitter 5, and an apparatus main unit 10. The apparatus main unit 10 is connected to an external device 6 via a network 100.

The ultrasound probe 1 includes multiple transducer elements 11 that generate ultrasound on the basis of drive signals supplied from the transmitter/receiver 11 of the apparatus main unit 10. The transducer elements of the ultrasound probe 1 are, for example, piezoelectric transducer elements. The ultrasound probe 1 receives reflected wave signals from a patient P and converts them to electric signals. The ultrasound probe 1 has matching layers provided to the piezoelectric transducer elements and backing members for preventing backward propagation of ultrasound from the transducer elements. The ultrasound probe 1 is detachably connected to the apparatus main unit 10.

When ultrasound is transmitted from the ultrasound probe 1 to the patient P, the transmitted ultrasound is sequentially reflected on the discontinuous plane of acoustic impedance in a body tissue of the patient P and is received as reflected wave signals by the multiple transducer elements of the ultrasound probe 1. The amplitude of the received reflected wave signals depends on the difference in acoustic impedance on the discontinuous plane. The reflected wave signals resulting from reflection of transmitted ultrasound pulses on the surface of the moving blood flow, the surface of the cardiac wall, etc. undergo, due to Doppler effect, a frequency shift depending on the velocity component with respect to the ultrasound transmission direction in a mobile object.

For example, for two-dimensional scanning of the patient P, a 1D array probe having multiple piezoelectric transducer elements arranged in a line is connected as the ultrasound probe 1 to the apparatus main unit 10. The 1D array probe serving as the ultrasound probe 1 is, for example, a sector probe for performing sector scanning, a convex probe for performing offset sector scanning, a linear probe for performing linear scanning, etc.

Alternatively, for example, for three-dimensional scanning of the patient P, a mechanical 4D probe or a 2D array probe is connected to the apparatus main unit 10 as the ultrasound probe 1. A mechanical 4D probe is capable of two-dimensional scanning using multiple piezoelectric transducer elements that are arrayed in a line as those of a 1D array probe and is capable of three-dimensional scanning by oscillating the multiple piezoelectric transducer elements by a given angle (oscillation angle). Furthermore, a 2D array probe is capable of three-dimensional scanning using multiple transducer elements arrayed in matrix and is capable of two-dimensional scanning by transmitting focused ultrasound.

The position sensor 4 and the transmitter 5 are devices for acquiring the positional information on the ultrasound probe 1. For example, the position sensor 4 is a magnetic sensor that is attached to the ultrasound probe 1. In addition, for example, the transmitter 5 is a device that is arranged in an arbitral position and forms a magnetic field outward about the transmitter 5.

The position sensor 4 detects a three-dimensional magnetic field that is formed by the transmitter 5. The position sensor 4 then calculates the position (coordinates and angle) of the position sensor 4 in the space using the transmitter 5 as its origin and transmits the calculated position to a controller 17 to be described below. The position sensor 4 transmits the three-dimensional coordinates and angle of the position of the position sensor 4 as three-dimensional positional information on the ultrasound probe 1 to the controller 17 to be described below.

The input device 3 is interfaced with the apparatus main unit 10 via an interface unit 18 to be described below. The input device 3 includes a mouse, a keyboard, buttons, a panel switch, a touch command screen, a fit switch, a track ball, etc. The input device 3 accepts various types of setting requests from an operator of the ultrasound diagnostic apparatus and transfers the accepted various types of setting requests to the apparatus main unit 10.

The monitor 2 is a display device that displays a GUI (Graphical User Interface) for the operator of the ultrasound diagnostic apparatus to input various types of setting requests using the input device 3 and that displays ultrasound image data that is generated by the apparatus main unit 10.

The external device 6 is a device that is interfaced with the apparatus main unit 10 via the interface unit 18 to be described below. For example, the external device 6 is a database of a PACS (Picture Archiving and Communication System) that is a system that manages various types of medical image data, a database of an electronic health record system that manages electronic health records attached with medical images, etc. Alternatively, the external device 6 is, for example, one of various types of medical image diagnosis apparatuses other than the ultrasound diagnostic apparatus according to the embodiments, such as an X-ray CT (Computed Tomography) apparatus, an MRI (Magnetic Resonance Imaging) apparatus, etc. Alternatively, the external device is, for example, a PC (Personal Computer) used by a doctor who performs image diagnosis, a recording medium such as a CD or DVD, a printer, etc.

The apparatus main unit 10 according to the embodiment can acquire data of various types of medical images that are uniformed into an image format according to DICOM (Digital Imaging and Communications in Medicine) from the external device 6 via the interface unit 18. For example, the apparatus main unit 10 can acquire, via the interface unit 18 to be described below, volume data to be compared to ultrasound image data that is generated by the apparatus main unit 10 from the external device 6 via the interface unit 18.

The apparatus main unit 10 is a device that generates ultrasound image data on the basis of the reflected wave signals received by the ultrasound probe 1. The apparatus main unit 10 shown in FIG. 1 is a device capable of generating two-dimensional ultrasound image data on the basis of two-dimensional reflected wave signals and capable of generating three-dimensional ultrasound image data on the basis of three-dimensional reflected wave signals.

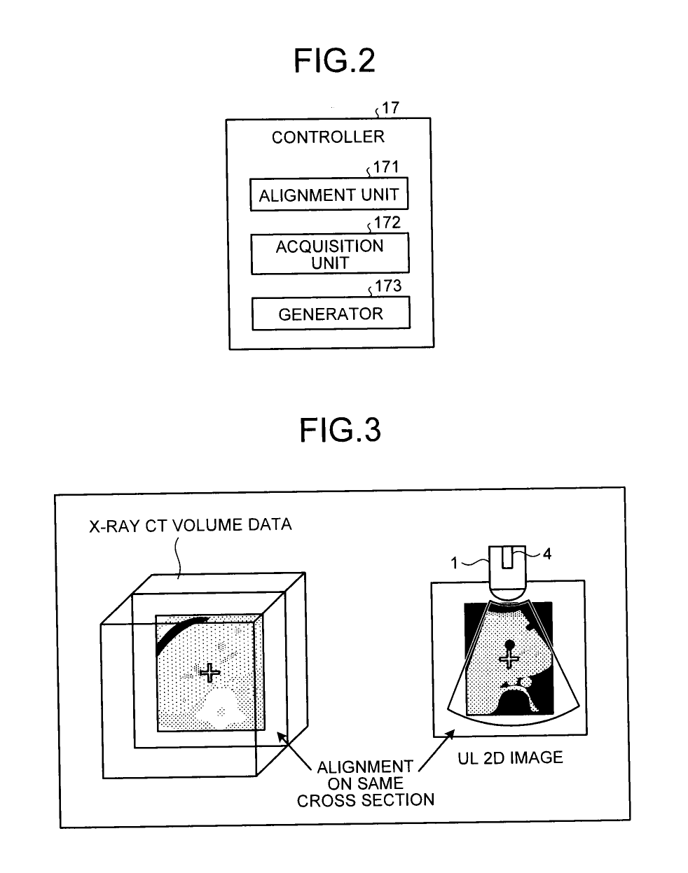

The apparatus main unit 10 includes, as shown in FIG. 2, the transmitter/receiver 11, a B-mode processor 12, a Doppler processor 13, an image generator 14, an image memory 15, an internal storage unit 16, the controller 17, and an interface unit 18.

The transmitter/receiver 11 controls transmitting/receiving of ultrasound performed by the ultrasound probe 1. The transmitter/receiver 11 includes a pulse generator, a transmission delay unit, a pulsar, etc. and supplies drive signals to the ultrasound probe 1. The pulse generator repeatedly generates rate pulses for forming transmission ultrasound at a given rate frequency. The transmission delay unit focuses the ultrasound generated from the ultrasound probe 1 into beams and gives, to each rate pulse generated by the pulse generator, a delay time per piezoelectric transducer element that is necessary to determine the transmission directionality. The pulsar applies a drive signal (drive pulse) to the ultrasound probe 1 at a timing based on the rate pulse. The transmission delay unit changes the delay time given to each rate pulse so as to arbitrarily adjust the direction in which the ultrasound transmitted from the surface of the piezoelectric transducers is transmitted.

The transmitter/receiver 11 has a function capable of instantly changing the transmission frequency, transmission drive voltage, etc. in order to execute a given scanning sequence according to an instruction of the controller 17 to be described below. Particularly, changing the transmission drive voltage is implemented by using a linear-amplifier outgoing circuit capable of instantly switching the value of voltage or a mechanism that electrically switches on/off multiple power units.

The transmitter/receiver 11 includes a preamplifier, an A/D (Analog/Digital) converter, a receiving delay unit, an adder, etc. and generates reflected wave data by performing various processes on the reflected wave signals received by the ultrasound probe 1. The preamplifier amplifies reflected wave signals on a channel basis. The A/D converter performs A/D conversion on the amplified reflected wave signals. The receiving delay unit gives a delay time necessary to determine receiving directionality. The adder performs an add process on the reflected wave signals processed by the receiving delay unit to generate reflected wave data. The add process performed by the adder intensifies the reflected components from the direction corresponding to the receiving directionality of reflected wave signals and synthetic beams of transmitting/receiving ultrasound is formed according to the receiving and transmitting directionality.

When two-dimensionally scanning the patient P, the transmitter/receiver 11 causes the ultrasound probe 1 to transmit two-dimensional ultrasound beams. The transmitter/receiver 11 then generates two-dimensional reflected wave data from two-dimensional reflected wave signals received by the ultrasound probe 1. When the transmitter/receiver 11 three-dimensionally scans the patient P, the transmitter/receiver 11 causes the ultrasound probe 1 to transmit three-dimensional ultrasound beams. The transmitter/receiver 11 then generates three-dimensional reflected wave data from the three-dimensional reflected wave signals received by the ultrasound probe 1.

For the mode of output signals from the transmitter/receiver 11 can be selectable from various modes, such as signals containing phase information referred to as RF (Radio Frequency) signals, amplitude information after envelope demodulation processing, etc.

The B-mode processor 12 and the Doppler processor 13 are signal processors that perform various types of signal processing on reflected wave data that is generated by the transmitter/receiver 11 from the reflected wave signals. The B-mode processor 12 receives reflected wave data from the transmitter/receiver 11 and performs logarithmic amplification, envelope demodulation processing, etc. to generate data (B-mode data) expressing the signal intensity by luminance brightness. The Doppler processor 13 analyzes the frequency of the velocity information from the reflected wave data received from the transmitter/receiver 11 and generates data (Doppler data) obtained by extracting moving object information, such as velocity, dispersion and power, with respect to many points. Here, the moving object is, for example, the blood flow, tissues such as the cardiac wall, and a contrast agent.

The B-mode processor 12 and the Doppler processor 13 illustrated in FIG. 1 are capable of processing both of two-dimensional reflected wave data and three-dimensional reflected wave data. In other words, the B-mode processor 12 generates two-dimensional B-mode data from two-dimensional reflected wave data and generates three-dimensional B-mode data from three-dimensional reflected wave data. The Doppler processor 13 generates two-dimensional Doppler data from two-dimensional reflected wave data and generates three-dimensional Doppler data from three-dimensional reflected wave data.

The image generator 14 generates ultrasound image data from data that is generated by the B-mode processor 12 and the Doppler processor 13. In other words, the image generator 14 generates two-dimensional B-mode image data representing the intensity of reflected waves by luminance from the B-mode data generated by the B-mode processor 12. The image generator 14 generates two-dimensional Doppler image data representing the moving-object information from the two-dimensional Doppler data generated by the Doppler processor 13. The two-dimensional Doppler image data is velocity image data, dispersion image data, power image data, or image data that is a combination thereof.

The image generator 14 converts a sequence of scanning line signals of ultrasound scanning to a sequence of scanning line signals in a video format known by TV (scan conversion) etc. and generates ultrasound image data to be displayed. Specifically, by performing coordinate conversion according to the mode of scanning using ultrasound performed by the ultrasound probe 1, the image generator 14 generates ultrasound image data to be displayed. The image generator 14 performs, as various types of image processing other than scan conversion, for example, image processing (smoothing processing) for regenerating a luminance-value averaged image using multiple image frames after scan conversion, image processing (edge enhancement process) using a differential filter in an image, etc. The image generator 14 combines additional information (letters information about various parameters, scales, body marks etc.) with ultrasound image data.

In other words, B-mode data and Doppler data are ultrasound image data before the scan conversion process and the data generated by the image generator 14 is ultrasound image data after the scan conversion process that is to be displayed. The B-mode data and Doppler data are also referred to as raw data. The image generator 14 generates “two-dimensional B-mode image data and two-dimensional Doppler image data” that are two-dimensional ultrasound image data to be displayed from “two-dimensional B-mode data and two-dimensional Doppler data” that is two-dimensional ultrasound image data before the scan conversion process.

Furthermore, the image generator 14 generates three-dimensional B mode image data by performing coordinate conversion on three-dimensional B-mode data generated by the B-mode processor 12. The image generator 14 generates three-dimensional Doppler image data by performing coordinate conversion on three-dimensional Doppler data generated by the Doppler processor 13. The image generator 14 generates “three-dimensional B-mode image data and three-dimensional Doppler image data” as “three-dimensional ultrasound image data (ultrasound volume data)”.

The image generator 14 performs a rendering process on the volume data in order to generate various type of two-dimensional image data for displaying the volume data on the monitor 2. As the rendering process performed by the image generator 14, there is a process for performing MPR (Multi Planar Reconstruction) to generate MPR image data from the volume data. Furthermore, as the rendering process performed by the image generator 14, for example, there is a VR (Volume Rendering) process to generate two-dimensional image data reflecting three-dimensional information.

By using the rendering function of the image generator 14, the ultrasound diagnostic apparatus according to the embodiment displays VE (virtual endoscopy) image data using ultrasound volume data containing luminal tissues. The VE image data is image data generated from volume data by perspective projection using the viewpoint and the line of sight set in the lumen. The image generator 14 displays, as video images, VE image data of different viewpoints by shifting the viewpoint along the center line (core line) of the lumen. When this video display is performed, the inner wall of the lumen serves as a clip area to be rendered. However, because of its nature, the ultrasound diagnostic apparatus is not suitable for observation of internal organs, such as the digestive organs not filled with water or substances. Thus, application of video image display performed by the ultrasound diagnostic apparatus covers the lumen that is filled with fluid, such as blood vessels filled with blood and the binary duct filled with bile.

The image memory 15 is a memory that stores the image data to be displayed, which is generated by the image generator 14. The image memory 15 is capable of storing data that is generated by the B-mode processor 12 and the Doppler processor 13. The B-mode data and Doppler data that the image memory 15 stores can be, for example, called by the operator after diagnosis, and it will become, via the image generator 14, ultrasound image data to be displayed.

The internal storage unit 16 stores various types of data such as a control program for performing transmitting/receiving ultrasound, image processing, and display processing, diagnostic information (e.g., patient IDs, doctor's opinions, etc.), diagnostic protocols, and various body marks. The internal storage unit 16 is also used for storing the image data that is stored by the image memory 15 if required. The data stored by the internal storage unit 16 can be transferred to the external device 6 via the interface unit 18 to be described below.

The controller 17 controls whole processes performed by the ultrasound diagnosing apparatus. Specifically, on the basis of various setting requests that are input by the operator via the input device 3 and various control programs and various types of data that are read from the internal storage unit 16, the controller 17 controls processes performed by the transmitter/receiver 11, the B-mode processor 12, the Doppler processor 13 and the image generator 14. The controller 17 further performs control such that the image data to be displayed, which is generated by the image generator 14, is stored in the internal storage unit 16 etc. The controller 17 further performs control such that medical image data that is accepted from the operator via the input device 3 is transferred from the external device 6 to the internal storage unit 16 and the image generator 14 via the network 10 and the interface unit 18.

The interface unit 18 is an interface for the input device 3, the network 100 and the external device 6. Various types of setting information and various instructions from the operator that are accepted by the input device 3 are transferred via the interface unit 18 to the controller 17. For example, the interface unit 18 lets the external device 6 be notified, via the network 100, of a request from the operator for transferring the image data accepted by the input device 3. The interface unit 18 lets the image data be transferred by the external device 6 be stored in the internal storage unit 16 and be transferred to the image generator 14.

Transmitting/receiving data to/from the external device 6 via the interface unit 18 allows the controller 17 according to the embodiment to display, with the ultrasound images captured by the medical image diagnostic apparatus, medical images (X-ray CT images, MRI images, etc.) captured by another medical image diagnostic apparatus on the monitor 2. The medical image data to be displayed together with the ultrasound images may be stored in the internal storage unit 16 via a storage medium, such as a CD-ROM, an MO, and a DVD.

The controller 17 further causes the image generator 14 to generate medical image data on an approximately the same cross section as that of the two-dimensional ultrasound image data displayed on the monitor 2 and causes the monitor 2 to display it. Here, the cross section of the two-dimensional ultrasound image data displayed on the monitor 2 is, for example, a cross section of two-dimensional ultrasound scanning that is performed to generate two-dimensional ultrasound image data, a cross section of two-dimensional ultrasound scanning that is performed to determine an area for three-dimensional ultrasound scanning for acquiring ultrasound volume data, or a cross section corresponding to cross-sectional image data (MPR image data etc.) that is generated from ultrasound volume data. For example, when performing ultrasound examination on the patient P, the operator issues a request for transferring X-ray CT volume data obtained by imaging a target site of the patient P to be examined. The operator further adjusts the position of the cut plane for MPR processing via the input device 3 such that the X-ray CT image data depicting the target site is displayed on the monitor 2.

Under the control of the controller 17, the image generator 14 generates X-ray CT image data obtained by cutting the X-ray CT volume data along a cut plane that is adjusted by the operator (hereinafter, “initial cross section”), and the monitor 2 displays the two-dimensional X-ray CT image data that is generated by the image generator 14. The operator operates the ultrasound probe 1 so as to perform ultrasound scanning using the same plane as that of the X-ray CT image data displayed on the monitor 2. The operator readjusts the position of the initial cross section on the X-ray CT volume data so as to display an X-ray CT image of the same cross section as that of the ultrasound image data displayed on the monitor 2. When the operator determines that the cross section of the X-ray CT image data displayed on the monitor 2 and that of the ultrasound image data are approximately the same, the operator pushes an enter button of the input device 3. The controller 17 sets, as initial positional information, the three-dimensional positional information on the ultrasound probe 1 acquired from the position sensor 4 at the time when the enter button is pushed. Furthermore, the controller 17 determine, as a final initial cross section, the position of the initial cross section on the X-ray CT volume data at the time when the enter button is pushed.

The controller 17 then acquires shift information about the scanning plane of the ultrasound probe 1 from the three-dimensional positional information and initial positional information on the ultrasound probe 1 that are acquired from the position sensor 4 and changes the position of the initial cross section on the basis of the acquired shift information, thereby resetting a cut cross section for MPR. Under the control of the controller 17, the image generator 14 generates X-ray CT image data from the X-ray CT volume data by using the cut cross section that is reset by the controller 17 and then generates image data where the X-ray CT image data and the ultrasound image data are parallelized. The monitor 2 displays the image data. Accordingly, the ultrasound diagnostic apparatus according to the embodiment can display an ultrasound image and an X-ray CT image of approximately the same cross section as that of the ultrasound image concurrently in real time. Hereinafter, the function of displaying an ultrasound image and an X-ray CT image etc. of the same cross section on the screen of the monitor 2 concurrently in real time can be referred to as “concurrent display function”.

An overall configuration of the ultrasound diagnostic apparatus according to the first embodiment is described above. Under such a configuration, the ultrasound diagnostic apparatus according to the first embodiment displays VE image data. The outline of structures in B-mode image data tends to be blurred compared to other medical images, such as X-ray CT images and MRI images. For this reason, for example, unless the lumen has a certain diameter or more, it is difficult to detect the luminal area of the lumen from B-volume data by automatic processing using a program. Particularly, in the case of blood vessels with strong movement due to pulsation, the outline of blood vessels further tends to be blurred. Thus, under the circumstances, unless the lumen has the certain thickness or more, a clip area cannot be detected. For this reason, display of VE image data by conventional ultrasound diagnostic apparatuses is limited to tubular tissues having the certain thickness and is difficult to be applied to narrow tubular tissues.

Thus, in the ultrasound diagnostic apparatus according to the first embodiment, in order to acquire the outline of structures depicted in an ultrasound image, the process of the controller 17 described below is performed. Specifically, the controller 17 according to the first embodiment performs the process described below in order to acquire the outline of structures depicted in an ultrasound image and display VE image data even of narrow tubular tissues.

The process performed by the controller 17 according to the first embodiment will be described below using FIG. 2. FIG. 2 is a diagram for describing an exemplary configuration of the controller 17 according to the first embodiment. As shown in FIG. 2, the controller 17 includes an alignment unit 171, an acquisition unit 172, and a generator 173.

The alignment unit 171 performs alignment between ultrasound image data and different-type medical image data of a type other than the ultrasound image data. For example, the alignment unit 171 accepts specifying of two sets of volume data where ultrasound image data is three-dimensional ultrasound volume data and different-type medical image data is three-dimensional different-type medical image volume data as well as accepts a request for displaying VE image data. The alignment unit 171 performs alignment between the specified two sets of volume data.

The alignment unit 171 according to the first embodiment performs alignment using the above-mentioned “concurrent display function” as an example. Alignment between ultrasound volume data and X-ray CT volume data that is different-type medical image volume data that is performed by the alignment unit 171 will be described below using FIG. 3. FIG. 3 is a diagram for describing the alignment unit according to the first embodiment. First, the operator issues a request for transferring X-ray CT volume data obtained by imaging a target site containing the lumen of the patient P to be displayed on VE image data. The alignment unit 171 thus acquires the X-ray CT volume data to be aligned as shown in FIG. 3. The operator further performs three-dimensional ultrasound scanning for acquiring ultrasound volume data containing the lumen of the patient P to be displayed on VE image data.

For example, the operator uses the ultrasound probe 1 capable of three-dimensional ultrasound scanning to perform two-dimensional ultrasound scanning of the patient P on a given cross section. Here, the given cross section is set, for example, as a cross section positioned at the center of a three-dimensional area where three-dimensional ultrasound scanning is performed. Because the controller 17 controls receiving of ultrasound via the transmitter/receiver 11, it can acquire the relative position of the cross section with respect to the ultrasound probe 1.

The operator then operates the ultrasound probe 1 attached with the position sensor 4 with reference to the ultrasound image (UL2D image shown in FIG. 3) displayed on the monitor 2 such that the target site is depicted at approximately the center of the ultrasound image. The operator also adjusts the position of the cut cross section for MPR processing via the input device 3 such that the X-ray CT image data depicting the target site is displayed on the monitor 2.

When the same feature part as that of the target site depicted on the MRP image of the X-ray CT volume data is depicted on the UL2D image, the operator pushes the enter button. The operator specifies the center position of the featuring part in each image with a mouse. Alternatively, the operator specifies multiple positions of a feature part in each image with a mouse. The operator then performs three-dimensional ultrasound scanning on the patient P in the three-dimensional area containing the two-dimensional ultrasound scanning cross section at the time when the enter button is pushed. Accordingly, the image generator 14 generates ultrasound volume data. The alignment unit 171 performs alignment between X-ray CT volume data and ultrasound volume data according to the cut cross section of the X-ray CT volume data, the three-dimensional positional information on the ultrasound probe 1, and the position of the feature site in each of the UL2D image and the CTMPR image at the time when the enter button is pushed.

In other words, the alignment unit 171 associates the coordinates of the voxel of the X-ray CT volume data and the coordinates of the voxel of the ultrasound volume data according to the cut cross section of the X-ray CT volume data, the three-dimensional positional information on the ultrasound probe 1, and the position of the feature site of each of the UL2D image and the image at the time when the enter button is pushed. The process is performed so that, for example, even if the position of the ultrasound probe 1 is shifted and new ultrasound volume data is generated, the alignment unit 171 can perform alignment between the ultrasound volume data and the X-ray CT volume data. The method employed by the alignment unit 171 to perform alignment is not limited to the above method and, for example, it may be performed by employing a known technology such as alignment using a cross correlation method, etc.

The acquisition unit 172 specifies the position of a body tissue in the different-type medical image data and acquires the specified position of the body tissue on the ultrasound image data on the basis of the result of alignment. The acquisition unit 172 specifies, for example, the position of the luminal area as the position of the body tissue on the different-type medical image volume data. The acquisition unit 172 is an example of the detector.

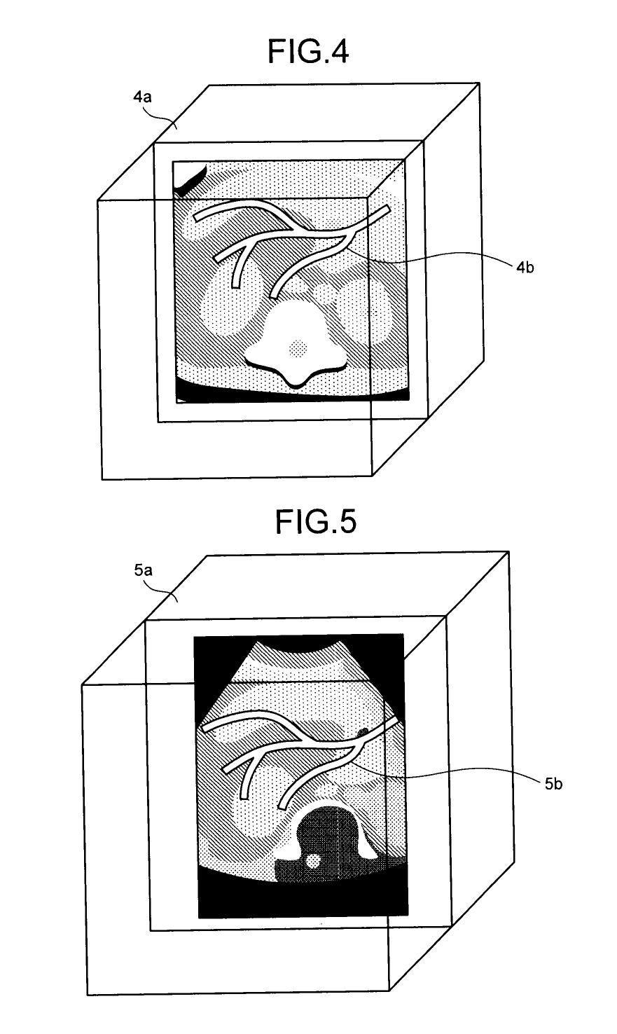

FIGS. 4 and 5 are diagrams for describing the acquisition unit according to the first embodiment. As shown in FIG. 4, the acquisition unit 172 extracts each area by performing, on X-ray CT volume data 4a on which alignment is performed by the alignment unit 171, segmentation processing using a pattern matching method using a region growing method for extracting an area where the CT value is spatially continuous and a shape template.

As shown in FIG. 4, with respect to extracted each area, the acquisition unit 172 then specifies and acquires the position of a blood vessel area 4b contained in the X-ray CT volume data 4a by employing a pattern matching method using a shape template for blood vessel area, a method of using the profile of the luminance of the blood vessel area, etc.

As shown in FIG. 5, the acquisition unit 172 acquires the position of the blood vessel area 4b on ultrasound volume data 5a on the basis of the result of alignment. As described above, the alignment unit 171 acquires the correspondence relationship between the coordinates of the voxel of the X-ray CT volume data 4a and the coordinates of the voxel of the ultrasound volume data. By using the correspondence relationship and from the position of the blood vessel area 4b on the X-ray CT volume data 4a, the acquisition unit 172 acquire the position of a blood vessel area 5b corresponding to the blood vessel area 4b on the ultrasound volume data 5a.

The generator 173 generates, as display image data to be displayed on the monitor 2, image data to which the position of the body tissue acquired by the acquisition unit 172 is reflected. The generator 173 processes the ultrasound image data on the basis of the position of the body tissue acquired by the acquisition unit 172 and generates, as display image data to be displayed on a given display unit, image data generated on the basis of the processed ultrasound image data.

Specifically, on the basis of the position of the luminal area that is acquired by the acquisition unit 172, the generator 173 generates, as display image data, projection image data obtained by projecting the ultrasound volume data from a viewpoint that is set in the luminal area. The generator 173 then performs processing to replace the voxel value in the blood vessel area 5b corresponding to the blood vessel area 4b by 0. In other words, the generator 173 perform processing to change the voxel value in the blood vessel area 5b corresponding to the blood vessel area 4b to 0. The generator 173 then generates, as image data to be displayed on the monitor 2, VE image data obtained by projecting the ultrasound volume data 5a with the voxel value replaced by 0 from the viewpoint that is set in the blood vessel area 5b.

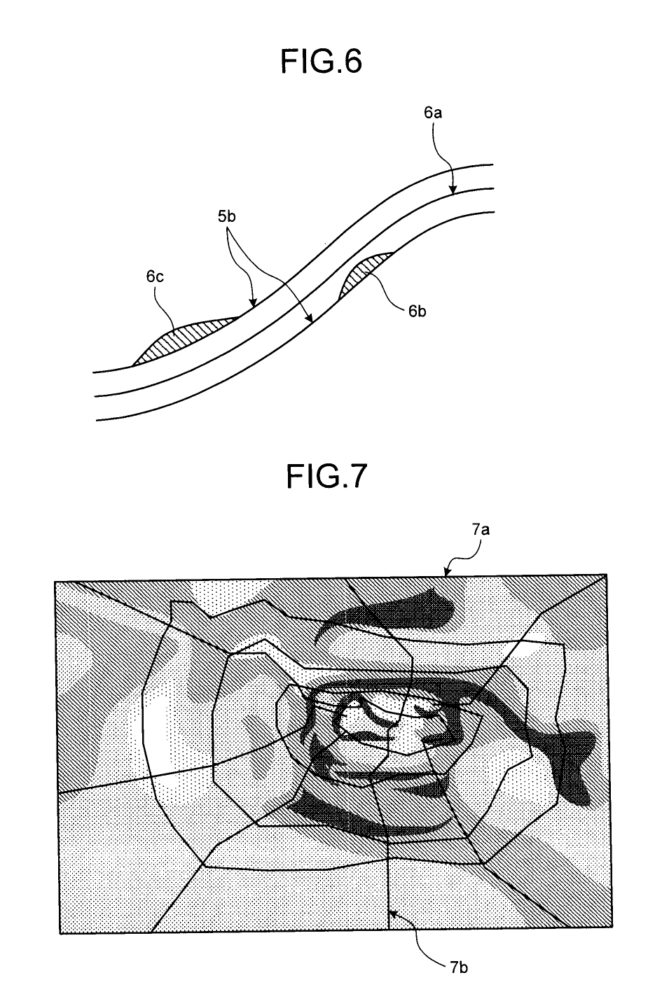

FIGS. 6 and 7 are diagrams for describing a generator according to the first embodiment. For example, as shown in FIG. 6, the generator 173 extracts a center line 6a of the blood vessel area 5b. The generator 173 then, as shown in FIG. 6, generates VE image data using the viewpoint that is set along the center line 6a. By shifting the viewpoint along the center line 6a, the generator 173 sequentially generates VE image data 7a to be displayed as video images, which is illustrated in FIG. 7. The generator 173 outputs the generated VE image data 7a to be displayed as video images to the monitor 2 and the monitor 2 displays the VE image data 7a as video images.

The generator 173 may generate the image data described below. For example, the generator 173 generates image data indicating the position the luminal area acquired by the acquisition unit 172 and generates, as display image data, the image data where the generated image data and projection image data are superimposed. For example, as depicted in FIG. 7, the generator 173 generates wire frame image data 7b indicating the boundary of the blood vessel area 5b acquired by the acquisition unit 172. The generator 173 then generates, as display image data to be displayed on the monitor 2, image data where the wire frame image data 7b is superimposed on the generated VE image data 7a. By referring to the image illustrated in FIG. 7, the operator can visually check the outline of the blood vessel area 5b corresponding to the blood vessel area 4b used for the VE image data 7a. The wire frame image data 7b illustrated in FIG. 7 is only an example. For example, the generator 173 may generate the surface of the luminal area as image data of a translucent tube and superimpose the generated image data on projection image data.

However, the blood vessel area 5b is an area corresponding to the blood vessel area 4b that is specified in the X-ray CT volume data 4a. For this reason, the outline of the blood vessel area 5b may not match the outline of the blood vessel area contained in the ultrasound volume data 5a. Thus, the generator 173 calculates the position of the luminal area on the ultrasound volume data and generates, as display image data, image data where an area corresponding to the difference between the calculated position and the position of the luminal area acquired by the acquisition unit 172 is displayed as highlighted. For example, the generator 173 acquires a voxel value of the ultrasound volume data 5a along the viewing direction from the viewpoint on the center line 6a that is set when generating the VE image data 7a. The generator 173 then, for example, regards the voxel of which voxel value is equal to or larger than a given threshold as a voxel corresponding to the inner wall of the blood vessel area on the ultrasound volume data 5a. Through the process, the generator 173 calculates the position of the blood vessel area on the ultrasound volume data.

The generator 173 then displays, as highlighted, an area corresponding to the difference between the calculated position of the blood vessel area on the ultrasound volume data 5a and the position of the blood vessel area 5b acquired by the acquisition unit 172. In the example shown in FIG. 6, the generator 173 generates image data where an upthrusting part 6b where the blood vessel area on the ultrasound volume data upthrusts into the blood vessel area 5b is displayed as highlighted. For example, the generator 173 uses, in the VE image data 7a, a red color as the color tone of the part corresponding to the upthrusting part 6b. The generator 173 also generates image data where a depressed part 6c where the blood vessel area on the ultrasound volume data is depressed outward with respect to the blood vessel area 5b is displayed as highlighted. For example, the generator 173 uses, in the VE image data 7a, a blue color as the color tone of the part corresponding to the depressed part 6c. As described, by displaying, as highlighted, a part where the outline of the blood vessel area contained in the ultrasound volume data 5a does not match the outline of the blood vessel area 5b, the operator can visually check that part easily. The highlighted display can be preformed concurrently with the display of the wire frame image data.

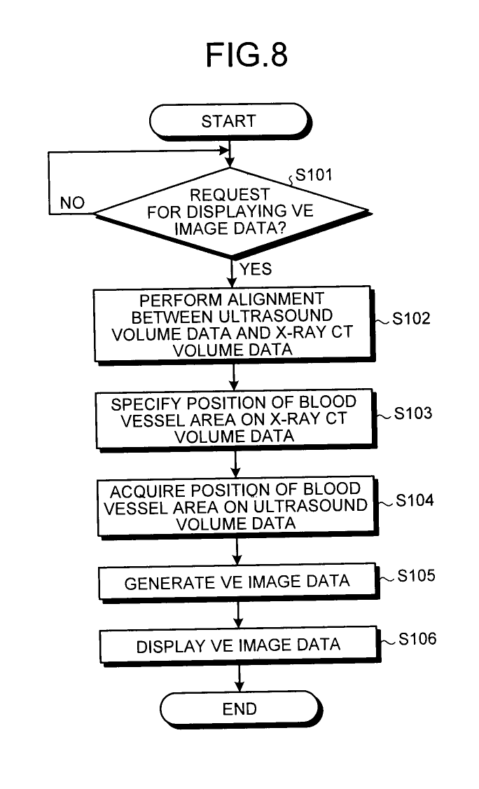

The process performed by the ultrasound diagnostic apparatus according to the first embodiment will be described using FIG. 8. FIG. 8 is a flowchart for describing an exemplary processing performed by the ultrasound diagnostic apparatus according to the first embodiment.

As shown in FIG. 8, when specifying of ultrasound volume data and X-ray CT volume data as well as a request for displaying VE image data are accepted (YES at step S101), the alignment unit 171 performs alignment between the ultrasound volume data and X-ray CT volume data (step S102). The alignment unit 171 is in a standby state until accepting specifying of ultrasound volume data and X-ray CT volume data as well as a request for displaying VE image data (NO at step S101).

The acquisition unit 172 specifies the position of the blood vessel area on the X-ray CT volume data (step S103) and acquires the specified position of the blood vessel area on the ultrasound volume data (step S104). The generator 173 generates VE image data by projecting the outline of the blood vessel area from a viewpoint that is set on the center line of the blood vessel area acquired by the acquisition unit 172 (step S105). The generator 173 outputs the generated VE image data to the monitor 2 and displays the VE image data on the monitor 2 (step S106). As an example, the generator 173 sequentially generate VE image data 7a to be displayed as video images and displays, as video images, the VE image data 7a to be displayed as video images. As another example, the generator 173 displays the generated VE image data as still images on the monitor 2.

As described above, the ultrasound diagnostic apparatus according to the first embodiment specifies the blurred outline of a structure on ultrasound image data by using a different-type medical image data of a type other than the ultrasound image. The ultrasound diagnostic apparatus then perform alignment between ultrasound image data and different-type medical image data to acquire, in the different-type medical image data, the position of the outline of the structures specified on the ultrasound image data. As described above, by using different-type medical image data after alignment, the ultrasound diagnostic apparatus can acquire the outline of the structure depicted in the ultrasound image.

Because the ultrasound diagnostic apparatus according to the first embodiment acquires the outline of a structure depicted in an ultrasound image, it can acquire the outline even of a narrow tubular tissue (blood vessel area etc.) that is difficult to be acquired from an ultrasound image. The ultrasound diagnostic apparatus acquires the center line from the acquired outline of a tubular tissue and projects the outline of the tubular tissue by using an arbitral point on the center line as the viewpoint, thereby generating VE image data. Thus, the ultrasound diagnostic apparatus enables display of VE image data even of a narrow tubular tissue as video images.

The ultrasound diagnostic apparatus according to the first embodiment generates wire frame image data indicating the position of the outline of the tubular tissue and displays it as superimposed on the ultrasound image data. Accordingly, the ultrasound diagnostic apparatus can let the operator to visually check the outline of the tubular tissue acquired from different-type medical image data.

The ultrasound diagnostic apparatus according to the first embodiment displays, as highlighted, a part where the outline of the tubular tissue contained in the ultrasound volume data does not match the outline of the tubular tissue specified from the different-type medical image data. Accordingly, the ultrasound diagnostic apparatus can let the operator to visually check easily the part where the outlines of the structure do not match to each other.

The first embodiment may be applied to a case where the above-described process performed by the generator 173 is performed by the image generator 14.

### Second Embodiment

While the first embodiment is described above, it may be carried out in various different modes other than the first embodiment.

(1) Display Mode Other than Virtual Endoscopic Display

In the first embodiment, the case is described where the position of an area on ultrasound volume data corresponding to a luminal area on different-type medical image volume data is acquired from the result of alignment between the ultrasound volume data and different-type medical image volume data and it is displayed using a virtual endoscope. However, embodiments are not limited to this. For example, the ultrasound diagnostic apparatus is capable of generating display image data in other display modes described below.

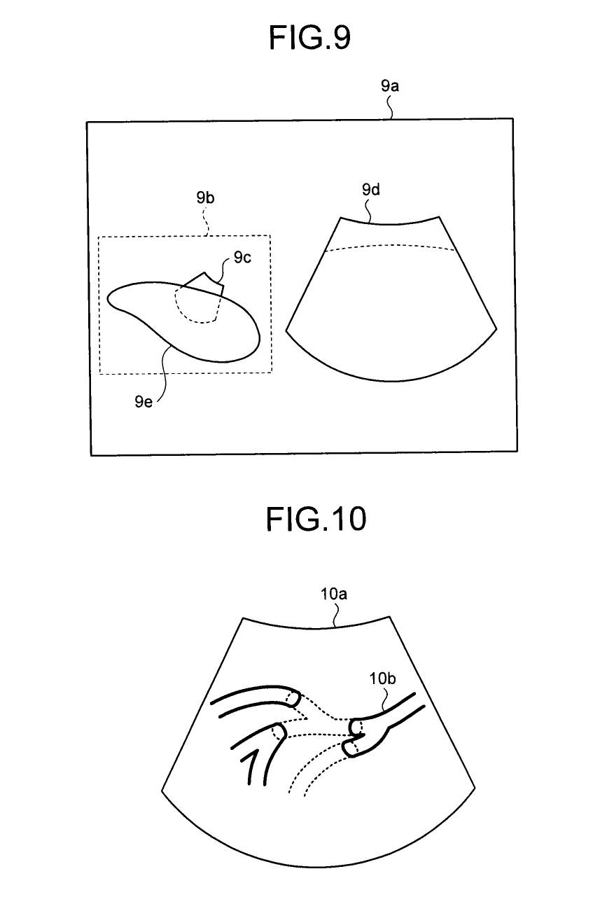

FIGS. 9 and 10 are diagrams depicting other exemplary display image data. FIG. 9 illustrates a case where the lever of a patient P is observed using two-dimensional ultrasound image data 9d. In FIG. 9, display image data that is generated as a result of alignment between two-dimensional ultrasound image data 9d obtained by imaging a part of the lever of the patient P and X-ray CT volume data obtained by capturing an image containing the entire lever of the patient P is displayed on a display area 9a of the monitor 2. First, the alignment unit 171 performs alignment between the two-dimensional ultrasound image data 9d and the X-ray CT volume data. The acquisition unit 172 then specifies the position of the lever contained in the X-ray CT volume data by segmentation processing. The acquisition unit 172 then acquires, in the two-dimensional ultrasound image data 9d, the position of an area corresponding to the lever on the X-ray CT volume data. The generator 173 then generates guide image data 9b illustrated in FIG. 9. The generator 173 then displays the guide image data 9b and the two-dimensional ultrasound image data 9d on the display area 9a. The position of the lever is specified as an area containing the outline of the lever as shown in FIG. 9.

The guide image data 9b shown in FIG. 9 is image data indicating the position of the lever on the cross section of scanning performed for generating the two-dimensional ultrasound image data 9d. The guide image data 9b is, as illustrated in FIG. 9, image data where scanning area image data 9c and lever image data 9e are superimposed. The generator 173 generates three-dimensional lever image data 9e by performing, on the lever contained in the X-ray CT volume data, the volume rendering process on the lever from the viewpoint that is set outside the lever. From the result of the alignment processing, the generator 173 generates the scanning area image data 9c where the area corresponding to the scanning area on the lever image data 9e is indicated by solid and dotted lines. The dotted line on the scanning area image data 9c indicates the scanned area in the lever and the solid line indicates the scanned area outside the lever. The guide image data 9b is reduced in size so as to be displayed on the display area 9a.

By referring to the guide image data 9b, the operator can know that the area where the scanning area image data 9c and the lever image data 9e are superimposed is depicted in the two-dimensional ultrasound image data 9d.

FIG. 10 illustrates a case where the blood vessel area of the patient P is observed using two-dimensional ultrasound image data 10a. In FIG. 10, display image data that is generated as a result of performing alignment between two-dimensional ultrasound image data 10a obtained by imaging a blood vessel area of the abdomen of the patient P and X-ray CT volume data obtained by imaging the blood vessel area of the abdomen of the patient P is displayed on the monitor 2. First, the alignment unit 171 performs alignment between the two-dimensional ultrasound image data 10a and the X-ray CT volume data. The acquisition unit 172 specifies the position of the blood vessel area contained in the X-ray CT volume data by segmentation processing. The acquisition unit 172 then acquires, in the two-dimensional ultrasound image data 10a, the position of an area corresponding to the blood vessel area on the X-ray CT volume data. The generator 173 then generates, as display image data, blood vessel schematic diagram data 10b illustrated in FIG. 10.

The blood-vessel schematic diagram data 10b depicted in FIG. 10 is image data indicating a stereoscopic relationship between the two-dimensional ultrasound image data 10a and the blood vessel area on the X-ray CT volume data. The generator 173 performs volume-rendering processing on the blood-vessel area contained in the X-ray CT volume data from the viewpoint that is set outside the blood vessel area. The generator 173 then generates the blood vessel schematic diagram data 10b by indicating, as a solid line, the outline of the area positioned in front of the scanning cross section of the two-dimensional ultrasound image data 10a and, as a dotted line, the outline of the area positioned behind the scanning cross section. Then, on the basis of the result of alignment processing, the generator 173 displays the blood-vessel schematic diagram data 10b as superimposed on the two-dimensional ultrasound image data 10a on the monitor 2.

Referring to the blood-vessel schematic diagram data 10b, the operator can know not only the blood-vessel area depicted on the two-dimensional ultrasound image data 10a but also the blood-vessel area not depicted on the two-dimensional ultrasound image data 10a together with the position on a three-dimensional space.

(2) Medical Image Processing Apparatus

The image processing method that is described in the above-described first embodiment and “Display Mode other than Virtual Endoscopic Display” may be performed by a medical image processing apparatus that is set independently of the ultrasound diagnostic apparatus. The medical image processing apparatus can receive ultrasound image data and different-type medical image data from a database of a PACS, a database of an electronic health record system, etc. and perform the above-described image processing method.

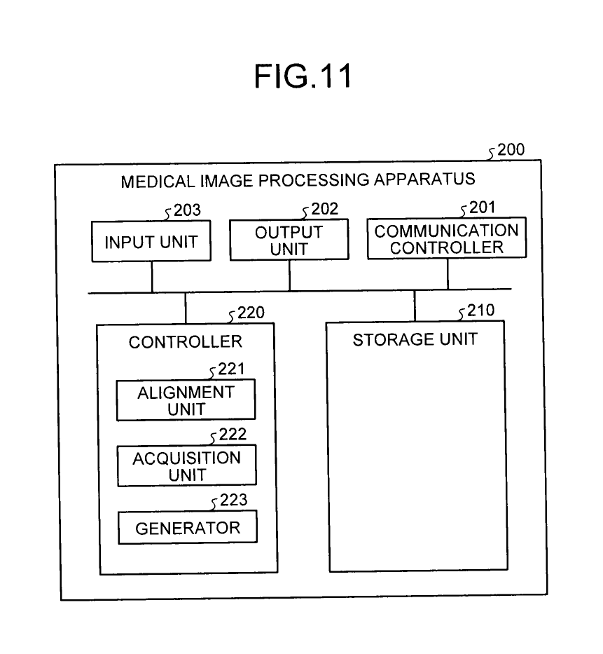

FIG. 11 is a block diagram depicting an exemplary configuration of a medical image processing apparatus according to a second embodiment. As shown in FIG. 11, a medical image processing apparatus 200 according to the second embodiment includes a communication controller 201, an output unit 202, an input unit 203, a storage unit 210, and a controller 220.

The communication controller 201 controls communications about various types of information received/transmitted between the medical image processing apparatus 200 and a database of a PACS, a database of an electronic health record system, etc. For example, the communication controller 201 receives ultrasound image data and different-type medical image data from the database of the PACS, the database of the electronic health record system, etc. For example, the communication controller 201 is a network interface card (NIC).

The output unit 202 is an output device that outputs various types of information. For example, the output unit 202 corresponds to a display, a monitor, etc.

The input unit 203 is an input device that accepts inputs of various types of information. For example, the input unit 203 accepts various setting requests from an operator of the medical image processing apparatus 200 and outputs the accepted various setting requests to the controller 220. For example, the input unit 203 corresponds to a keyboard, a mouse, etc.

The storage unit 210 stores various types of information. For example, the storage unit 210 corresponds to semiconductor memory devices such as a RAM (Random Access Memory) and a Flash Memory, and to storage devices such as a hard disk device and an optical disc device.

The controller 220 includes an alignment unit 221 having the same function as that of the alignment unit 171, an acquisition unit 222 having the same function as that of the acquisition unit 172, and a generator 223 having the same function as that of the generator 173. The function of the controller 220 can be implemented by, for example, an integrated circuit, such as an ASIC (Application Specific Integrated Circuit) or a FPGA (Field Programmable Gate Array). The function of the controller 220 can be also implemented by, for example, a CPU (Central Processing Unit) to execute a given program.

In the medical image processing apparatus 200, when the input unit 203 accepts specifying of ultrasound volume data and X-ray CT volume data as well as a request for displaying VE image data, the alignment unit 221 performs alignment between the ultrasound volume data and X-ray CT volume data. Subsequently, the acquisition unit 222 specifies the position of a blood-vessel area on the X-ray CT volume data and acquires the specified position of the blood vessel area on the ultrasound volume data. The generator 223 then generates VE image data by projecting the outline of the blood vessel area that is acquired by the acquisition unit 222 from the viewpoint that is set on the centerline of the blood vessel area. The generator 173 outputs the generated VE image data to the output unit 202 and causes it to display the VE image data.

As described above, the medical image processing apparatus 200 can receive ultrasound image data and different-type medical image data from the database of the PACS, the database of the electronic health record system, etc. and perform the above-described image processing method.

(3) Image Processing Program

The image processing method described in the above-described first embodiment and “(1) Display Mode other than Virtual Endoscopic Display” can be implemented in a way that the prepared image processing program is executed by a computer, such as a personal computer, a work station, etc. The image processing program can be distributed via a network, such as the Internet. The image processing program can be stored in a computer-readable non-temporary storage medium, such as a hard disk, a flexible disk (FD), a CD-ROM, an MO, a DVD, a Flash memory such as an USB memory or a SD card memory, and can be read by the computer from a non-temporal storage unit so as to be executed.

As described above, according to the first and second embodiments, the outline of a structured depicted on an ultrasound image can be acquired.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
an alignment unit that performs alignment between three-dimensional ultrasound volume data and three-dimensional different-type medical image volume data of a type other than the three-dimensional ultrasound volume data;
a detector that specifies the position of a luminal area on the different-type medical image volume data and detects the specified position of the luminal area on the ultrasound volume data; and
a generator that generates, as display image data to be displayed on a given display unit, projection image data obtained by projecting the ultrasound volume data from a viewpoint that is set in the luminal area on the basis of the position of the luminal area that is detected by the detector.

2. The ultrasound diagnostic apparatus according to claim 1, wherein the generator performs processing for changing the ultrasound volume data corresponding to the luminal area that is detected by the detector and generates, as the display image data, projection image data obtained by projecting the changed ultrasound volume data from the viewpoint that is set in the luminal area.

3. The ultrasound diagnostic apparatus according to claim 1, wherein the generator generates image data indicating the position of the luminal area that is detected by the detector and generates, as the display image data, image data where the image data and the projection image data are superimposed.

4. The ultrasound diagnostic apparatus according to claim 3, wherein the generator generates, as image data indicating the position of the luminal area that is detected by the detector, wire frame image data corresponding to the boundary of the luminal area and generates, as the display image data, image data where the wire frame image data and the projection image data are superimposed.

5. The ultrasound diagnostic apparatus according to claim 1, wherein the generator calculates the position of the luminal area on the ultrasound volume data on the basis of the projection image data and generates, as the display image data, image data where an area corresponding to the difference between the calculated position and the position of the luminal area that is detected by the detector is displayed as highlighted.

6. An ultrasound diagnostic apparatus comprising:
an alignment unit that performs alignment between ultrasound image data and different-type medical image data of a type other than ultrasound image data;
a detector that specifies an area containing the outline of a body tissue on the different-type medical image data and detects the position of the specified area on the ultrasound image data; and
a generator that generates, as display image data to be displayed on a given display unit, image data that is generated on the basis of the position of the body tissue that is detected by the detector and the ultrasound image data.

7. The ultrasound diagnostic apparatus according to claim 6, wherein the generator generates image data indicating the position of the outline that is detected by the detector and generates, as the display image data, image data where the image data and the projection image data are superimposed.

8. A medical image processing apparatus comprising:
an alignment unit that performs alignment between three-dimensional ultrasound volume data and three-dimensional different-type medical image volume data of a type other than the three-dimensional ultrasound volume data;
a detector that specifies the position of a luminal area on the different-type medical image volume data and detects the specified position of the luminal area on the ultrasound volume data; and
a generator that generates, as display image data to be displayed on a given display unit, projection image data obtained by projecting the ultrasound volume data from a viewpoint that is set in the luminal area on the basis of the position of the luminal area that is detected by the detector.

9. A medical image processing method comprising:
performing alignment between three-dimensional ultrasound volume data and three-dimensional different-type medical image volume data of a type other than the three-dimensional ultrasound volume data;
specifying the position of a luminal area on the different-type medical image volume data, and detecting the specified position of the luminal area on the ultrasound volume data; and
generating, as display image data to be displayed on a given display unit, projection image data obtained by projecting the ultrasound volume data from a viewpoint that is set in the luminal area on the basis of the position of the luminal area that is detected by the detector.
